(19) United States
(12) Patent Application Publication
(10) Pub. No.: US 2024/0033405 A1
(43) Pub. Date: Feb. 1, 2024

(54) VIBRATORY WAVEFORM FOR BREAST PUMP

(71) Applicant: LANSINOH LABORATORIES, INC., Alexandria, VA (US)

(72) Inventors: Rush Lloyd BARTLETT, II, Alexandria, VA (US); Frank Tinghwa WANG, Alexandria, VA (US)

(21) Appl. No.: 18/257,478

(22) PCT Filed: Dec. 9, 2021
(86) PCT No.: PCT/US2021/062639

§ 371 Date: Jun. 14, 2023

Related U.S. Application Data
(60) Provisional application No. 63/199,278, filed on Dec. 17, 2020.

(51) Int. Cl. A61M 1/06

(52) U.S. Cl. CPC A61M 1/0693; A61M 1/0697; A61M 2205/50

(57) ABSTRACT

Breast pumps allow for increased milk volume flow rates and/or increased pump efficiency. Various devices and techniques are used to introduce a more diverse set of vibration patterns which enable users to customize the performance of the vibration and/or enable vibrations to be present or absent in ways not currently enabled by the state of the art. This can involve applying vibrations to the breast during the breast pump cycle (or “waveform”), to increase the volume flow rate of expressed milk for a given cycle speed and suction level.

This application is being filed on Dec. 9, 2021, as a PCT International patent application and claims the benefit of and priority to U.S. Provisional Patent Application Ser. No. 63/199,278, filed Dec. 17, 2020, the entire disclosure of which is incorporated by reference in its entirety.

### CROSS-REFERENCE TO RELATED APPLICATIONS

This patent application is related to U.S. patent application Ser. No. 16/563,211, filed Sep. 9, 2019, entitled, “VIBRATORY WAVEFORM FOR BREAST PUMP.” The disclosure of this patent application is hereby incorporated by reference in its entirety into the present patent application.

### FIELD

The present application is directed to devices, systems and methods for facilitating the collection of breast milk.

### BACKGROUND

Breastfeeding is the recommended method to provide nutrients to a newborn child for the first year of life. Many mothers, however, return to work soon after giving birth, have difficulty breastfeeding their newborns, or have challenges breastfeeding for other reasons. As a result, many mothers rely on breast pumping to express their breast milk and use bottles to feed their newborns. Since a mother might need to pump as often as eight times a day to maintain her milk supply and/or prevent breast engorgement, it is essential that each breast pumping session be as efficient as possible—i.e., emptying as much milk from the breast as possible, in the shortest amount of time.

Breast pumps operate by applying a suction on the breast for a short period of time, during which a small amount of milk is expressed. The breast pump then releases the suction and repeats the cycle of on suction/off suction until the breast is empty. The amount of vacuum applied to the breast during one cycle of on suction/off suction, referred to as a waveform, is controlled by the breast pump by adjusting the applied voltage and/or current to an internal vacuum motor and solenoid, to mimic the baby feeding on the breast. Typical breast pumps allow the mother to adjust the cycle speed and the amount of suction, in an attempt to maximize efficiency of the pump. It is still often challenging, however, to adjust a breast pump to work efficiently.

Therefore, it would be ideal to have a breast pump that worked efficiently to prevent breast engorgement. Ideally, such a breast pump would empty as much milk from the breast as possible, in a short amount of time. Additionally, such a breast pump would also ideally be easy to adjust for an individual woman's specific needs. At least some of these objectives are addressed by the following disclosure.

### SUMMARY

The present document describes various devices and techniques to introduce a more diverse set of vibration patterns which enable users to customize the performance of the vibration and/or enable vibrations to be present or absent in ways not currently enabled by the state of the art.

This application describes an improved breast pump device and method that allows for increased milk volume flow rates and/or increased pump efficiency. The device and method involve applying vibrations to the breast during the breast pump cycle (or“waveform”), to increase the volume flow rate of expressed milk for a given cycle speed and suction level. The breast pump waveform with added vibrations according to the present disclosure is often referred to herein as a “vibratory waveform.” The vibratory waveform helps the breast pump empty milk from the breast more completely and/or in a shorter time than would occur from simply adjusting the breast pump's cycle speed and/or suction level. Creating a vibratory waveform may also reduce the time to let down, the reflex that leads to the release of breast milk. In any given pumping method example, the vibratory waveform may be applied, and the pump's cycle speed and/or suction level may also be adjusted. Alternatively, the vibratory waveform may be applied (and have advantageous results) without any adjustment of cycle speed or suction level.

In various embodiments, the breast pump applies vibration to the breast through small oscillations in the suction pattern as the vacuum is reduced, held, and/or released, as part of the pump cycle. The vibrations may facilitate improved let down and reduce the shear stress of milk against the inner walls of the milk ducts, to help increase the volume flow rate of milk flowing out of the milk duct. The vibration feeling is most pronounced when the suction is increased and decreased in a rapid cyclical manner.

The vibratory waveform can be generated in a breast pump system using a variety of devices and methods. In some embodiments, a vibratory device is added to a breast pump device. Alternatively, one or more components of a breast pump device may be altered or adjusted to cause vibrations. In other embodiments, a separate device may be used to generate vibrations. Examples of these types of embodiments include but are not limited to modulating the vacuum pump of a breast pump device, modulating the solenoid of a breast pump device, or adding a vibratory motor, a piezoelectric element, a speaker, a shaking element on the bottom of the pump motor housing or pump, an off-center rotary weight on the motor or shaft, or teeth in the wall of the piston housing that allow the diaphragm to “chatter” forward and backward. The vibration source can be built into the pump, the flange or an external device.

In one aspect of the present disclosure, a method for facilitating milk extraction from a female breast may involve applying a breast contacting portion of a breast pump system to a breast, activating the breast pump system to administer multiple breast pumping cycles, and applying vibrations to the breast during at least a portion of each of the breast pumping cycles, using a vibration device. In some embodiments, each of the breast pumping cycles may include an increasing vacuum segment, during which an amount of the vacuum force applied to the breast increases, and a decreasing vacuum segment, during which the amount of the vacuum force applied to the breast decreases.

Optionally, each of the breast pumping cycles may further include at least one vacuum hold segment, during which the amount of the vacuum force applied to the breast is held constant. For example, a vacuum hold segment may be a maximum vacuum force hold segment occurring after the increasing vacuum segment, during which the amount of the vacuum force is kept constant at a maximum vacuum force, or a minimum vacuum force hold segment occurring after the decreasing vacuum segment, during which the amount of the vacuum force is kept constant at a minimum vacuum force. Vibrations may be applied to any segment (or multiple segments) of the breast pumping cycle, including the increasing vacuum segment, the decreasing vacuum segment, and/or the vacuum hold segment(s). In some embodiments, the vibrations may be applied to the breast during an entire length of each cycle.

According to various embodiments, the applied vibrations may have a frequency of between 0 Hz and 10 MHz. More ideally, the vibrations may have a frequency of 5-10 Hz in some embodiments. Additionally, the frequencies of the vibration could change throughout the operation of the system to comprise frequencies inside and outside of this range. According to various embodiments, the vibrations may be applied in a pattern, such as but not limited to a stair-step pattern, a wavy pattern or an oscillating pattern.

In some embodiments, the vibration device that generates the vibrations in the breast is part of the breast pump system. Alternatively, the vibration device may be a separate device that is not directly connected to the breast pump system and that contacts the breast separately from the breast contacting portion of the breast pump system. For example, applying the vibrations may involve activating a motor and/or a solenoid that that is/are part of the vibration device. In some embodiments, applying the vibrations may involve applying an additional vacuum force via the breast pump system and releasing the additional vacuum force. For example, applying and releasing the additional vacuum force may involve driving air in an opposite direction through one or more holes in a one-way valve that is part of the breast pump system.

In some embodiments, the step of applying the vibrations is activated by a control unit of the breast pump system. Alternatively or additionally, applying the vibrations may be activated by a user of the breast pump system. Optionally, the method may further include adjusting the application of the vibrations. The adjusting may be performed by a control unit of the breast pump system and/or by a user, in various embodiments.

In another aspect of the present disclosure, a device for facilitating milk extraction from a female breast may include a housing and a vibration generating device coupled with the housing for creating vibrations in a breast to facilitate milk extraction from the breast. The device may be attached to, or incorporated into, a breast pump device. Alternatively, the device may be a separate device, used along with a breast pump device.

In some embodiments, the vibration generating device may be a motor. In some embodiments, the device is configured to directly contact the breast at a location apart from a breast pump device. Such a device may further include an adhesive surface on the housing for temporarily attaching the housing to the breast. The device may also optionally include a wireless module in the housing for transmitting signals to and/or receiving signals from a breast pump system.

In another aspect of the present disclosure, a system for facilitating milk extraction from a female breast may include a breast pump device and a vibration generating device. The breast pump device includes a breast contacting portion, a control unit with a vacuum source, and a connector for transmitting vacuum force from the vacuum source of the control unit to the breast contacting portion. The vibration generating device is coupled with the breast pump device for creating vibrations in a breast to facilitate milk extraction from the breast.

In some embodiments, the vibration generating device is attached to the breast contacting portion. In some embodiments, the vibration generating device is part of the control unit. In some embodiments, the vibration generating device is physically separate from the breast pump device and communicates with the breast pump device via wired or wireless communication. Different types of vibration generating devices include, but are not limited to, a motor, a stepper motor, a solenoid, a one-way valve with at least one hole, a piston, a weighted portion, and a software program in the control unit containing instructions to turn the vacuum force on and off. In some embodiments, the system may further include a controller for allowing a user of the system to adjust at least one parameter of the vibrations.

The control unit may include a number of different components, such as at least one motor, at least one solenoid, and electronics configured to control the motor and the solenoid. Some embodiments may include a first motor for providing the vacuum force to the breast contacting portion and a second motor for driving air into the breast contacting portion to generate the vibrations. In this example, the second motor is the vibration generating device. Some embodiments may include a flexible bulb coupled with the second motor, where the second motor squeezes and releases the flexible bulb to push air into and pull air out of the breast contacting portion.

These and other aspects and embodiments are described in greater detail below, in reference to the attached drawing figures.

### BRIEF DESCRIPTION OF THE DRAWINGS

FIG. 1 is a perspective view of a currently available electric breast pump system;

FIG. 2 is a time versus pressure diagram, showing a vibration applied via a breast pump by modulating a suction waveform along part of the suction induction breast pump curve, according to one embodiment;

FIG. 3 is a time versus pressure diagram, showing a vibration applied via a breast pump by modulating a suction waveform along all of the suction induction breast pump curve, according to an alternative embodiment;

FIG. 4 is a time versus pressure diagram, showing a vibration applied via a breast pump by modulating a suction waveform along the suction induction breast pump curve, except for a resting hold state at the lowest point in the vacuum, with no vibration or oscillation effect, according to one embodiment;

FIG. 5 is a time versus pressure diagram, showing a breast pump suction curve with a stair-step vibratory stimulation pattern of short stair-step bursts, according to one embodiment;

FIG. 6 is a time versus pressure diagram, showing a breast pump suction curve with alternating and/or independently modulated wave cycles, one including an oscillating effect and another including no oscillating effect, according to one embodiment;

FIG. 7 is a time versus pressure diagram, showing a breast pump suction curve including a drop-in pressure, a stair-step increase in pressure, and an additional cycle, with vibratory effects on at least part of the waveform curve segments, according to one embodiment;

FIGS. 8A-8D are time versus pressure diagrams that depict exemplary vibratory waveforms, each of which includes a vibration segment and a smooth segment during parts of the wave rise, fall, and/or hold segment(s), according to one embodiment;

FIG. 9 depicts a time versus pressure curve and exemplary motor and solenoid control signal curves, illustrating a modulating effect of the control signals on an oscillating pressure reduction curve from a breast pump suction waveform, according to one embodiment;

FIG. 10A is a graph showing a vacuum waveform with a stair-step vibration pattern, according to one embodiment;

FIG. 10B is a graph shoring a vacuum waveform with an oscillating increase and decrease vibration pattern, according to an alternative embodiment;

FIG. 11 illustrates a PCB, a pump motor, and a solenoid of a breast pump device, of which one or more may be used to drive activity of the breast pump waveform and waveform effects, according to various embodiments;

FIG. 12 is a side view of abreast pump flange and receptacle, with a vibration motor coupled with the breast pump flange, according to one embodiment;

FIG. 13 is a perspective view of a breast pump system including a breast pump flange and a separate vibration motor designed to be held by the user to mechanically vibrate the breast, according to one embodiment;

FIG. 14 is a side view of a breast pump flange with a moving membrane and an eccentric motor, according to one embodiment;

FIG. 15A is a side view of a vacuum motor device for providing a vibratory waveform to a breast pump, according to one embodiment;

FIG. 15B is a top view of a diaphragm of a one-way valve of the motor device of FIG. 15A, including multiple holes and with the flap of the valve removed to show the diaphragm;

FIG. 15C is a top view of the diaphragm of FIG. 15B, with the flap of the valve overlying the diaphragm and including a cutout portion to expose part of the diaphragm and one of the holes;

FIGS. 16A and 16B are side views showing operation of a conventional vacuum motor of breast pump system;

FIG. 17 is a side of the vacuum motor of FIG. 15A, illustrating operation of the motor to generate vibrations in the system, according to one embodiment;

FIG. 18 is a diagrammatic view of a breast pump system that includes a separate motor to generate a vibratory waveform, according to one embodiment;

FIG. 19 is a diagrammatic view of a breast pump system that includes a bulb the motor squeezes to increase pressure in the system, according to one embodiment;

FIG. 20 is a side view of another vacuum motor device for providing a vibratory waveform to a breast pump, according to one embodiment;

FIGS. 20A-20F are side views of alternative vacuum motor devices for providing vibrator waveforms to a breast pump, according to one embodiment;

FIGS. 21A-21C are side views of other alternative vacuum motor devices for providing vibratory waveforms to a breast pump, according to one embodiment;

FIGS. 21A-21C are side views of other alternative vacuum motor devices for providing vibratory waveforms to a breast pump, according to one embodiment;

FIGS. 22A-228 are side views of alternative vacuum motor devices for providing vibratory waveforms to a breast pump, according to one embodiment;

FIGS. 23A-23B are side views of alternative vacuum motor devices for providing vibratory waveforms to a breast pump, according to one embodiment;

FIG. 24 is a side view of another vacuum motor device for providing a vibratory waveform to a breast pump, according to one embodiment;

FIG. 25 is a side view of another vacuum motor device for providing a vibratory waveform to a breast pump, according to one embodiment;

FIGS. 26A-26C are side views of another alternative vacuum motor device for providing a vibratory waveform to a breast pump, according to one embodiment;

FIG. 27 is another time versus pressure diagram, showing another breast pump suction curve, according to one embodiment;

FIG. 28 is another time versus pressure diagram, showing another breast pump suction curve, according to one embodiment; and

FIG. 29 is yet another time versus pressure diagram, showing another breast pump suction curve, according to one embodiment.

### DETAILED DESCRIPTION

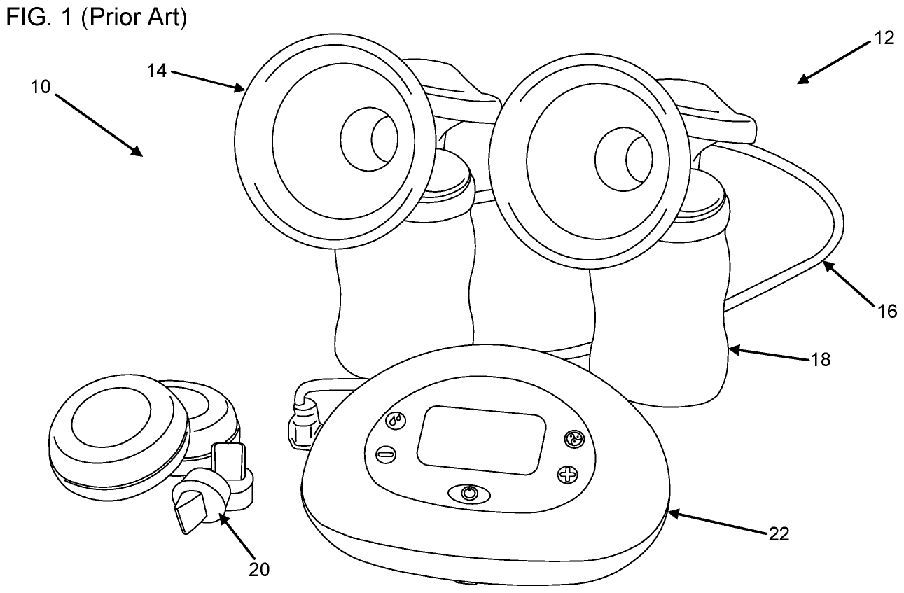

Referring to FIG. 1, one example of a currently available electric breast pump system 10 is shown. In this example, the system 10 includes a breast contacting portion 12 and a control unit 22. The breast contacting portion 12 typically includes two funnels 14 (or “shields”) for directly contacting and fitting partially over a woman's breasts, two milk collection receptacles 18 connected to the funnels 14, two duckbill valves 20 (or “membranes”) that reside inside the breast contacting portion 12 when in use, and a tube connector 16 for connecting the funnels 14 with the control unit 22. The control unit 22 typically includes several primary components, all of which are inside the housing of the control unit 22 and thus not visible in FIG. 1. For example, the control unit 22 typically houses a vacuum motor for generating vacuum (or “suction”) force that is conveyed through the tube connector 16 to the funnels 14, a solenoid that helps release vacuum pressure from the system 10, and electronics for driving the system 10.

Different terminology is sometimes used by people of skill in the art to refer to the various parts of a breast pump system 10. For example, the breast contacting portion may be referred to as a “milk extraction set” or a “disposable portion,” the funnels 14 are often referred to as “breast shields,” and the control unit is sometimes simply referred to as “the pump.” This application will typically use terminology as described immediately above, but these terms may in some cases be synonymous with other terms commonly used in the art. Therefore, the choice of terminology used to describe known components of a breast pump system or device should not be interpreted as limiting the scope of the invention as defined by the claims.

As mentioned in the Background section, currently available electric breast pump systems, such as the system 10 of FIG. 1, operate by applying vacuum force to the breast and releasing the vacuum force repeatedly during a pumping session. Each application and release of vacuum is referred to herein as one “cycle,” where each cycle begins as vacuum force starts to be applied and ends right before vacuum starts to be applied again. The pattern created on a graph of pressure versus time by an operating breast pump may be referred to herein as a “pumping waveform.”

Currently available breast pumps do not vibrate or generate vibrations in the breast as part of their regular function. Instead, they provide smooth, vibration-free suction and release cycles. In general, the methods described herein use one or more mechanisms to add vibrations to at least part of the breast pump cycle, in order to enhance the function of the breast pump and thus facilitate milk extraction from the breast. The application sometimes refers to the pumping waveform with the addition of vibrations as a “vibration waveform.” In other words, the “vibration waveform” may refer to any breast pumping waveform that has vibrations added to it.

Current breast pumps allow for changing the cycle speed and the suction pressure of the pump. The Hagen-Poiseuille fluid dynamic equation, derived from the approximation of a Newtonian fluid undergoing laminar flow, reads as follows: ΔP=(8μLQ)/(πR⁴), where ΔP=pressure difference (in the milk duct), L is length (of the milk duct), μ=dynamic viscosity (of the milk), Q=volumetric flow rate, and R=radius (of the milk duct). Current pumps only target ΔP by adjusting the suction pressure. Milk and colostrum can be approximated as a Newtonian fluid, and the dimension of the radius of the milk duct pipes can also enable us to be reasonably certain that almost all flow regimes encountered would consist of laminar flow segments. As a result, a Hagen-Poiseuille derivation from the shear stress equation τ=−μ* (dv/dr), where μ=viscosity, v=velocity of the fluid and r=the position along the radius in the tube, should represent a reasonable approximation. As such, the cycle speed of a breast pump affects how many suction and release cycles the breast pump operates in a minute but does not affect the volume flow rate during a cycle.

The devices, systems and methods described in this application enhance breast pump function by applying vibrations to reduce shear stress τ along a given radius of breast milk duct (or “conduit”), so that more volume in the duct will move at a higher velocity. The applied vibrations increase Q (volumetric flow rate) when other parameters are fixed and they may also stimulate the breast to induce let down and further increase the radial dimension R of the breast milk duct along critical flow restriction points. The decrease in μ from vibration may also be explained by the following equation, F=μA (ξ/y). With vibration, the friction between the fluid and the walls of the duct is decreased, thereby reducing the amount of force needed to maintain the flow velocity. In addition, or as a separate effect, vibration may stimulate let down, which increases the cross-sectional area of each milk duct. Going back to the Hagen-Poiseuille fluid dynamic equation, given a fixed ΔP, μ must decrease and Q must increase to balance the equation. Let down induces an increased radius and corresponding increase in Q, assuming the same pressure gradient.

The devices, systems and methods described herein use oscillation vibration patterns to induce increased milk flow from the breast during pumping, through one or more mechanical pathways. In various examples and embodiments, the devices, systems and methods may produce vibrations (or the vibratory waveform) with any suitable pattern, size, shape, timing, etc. For example, in any given embodiment, the frequency of the vibrations or oscillations may range from as low as just above 0 Hz as high as 10 MHz. There may be an ideal frequency range of the vibrations for comfort and the ability of the woman to feel the vibrations, which may for example be in a range of about 5 Hz to about 10 Hz. Alternatively, a wider range of about 2 Hz to about 20 Hz may be ideal in some embodiments. Generally, if the vibration frequency is too high, the woman will not feel the vibrations. On the other hand, high frequency vibration in the ultrasound range might be helpful in some instances, such as for unclogging milk ducts and alleviation of mastitis.

Just as any suitable type of vibrations may be applied, according to various embodiments, any suitable devices may be used to produce the vibrations, examples of which are described below. Therefore, this application should not be interpreted as being limited to any particular type or pattern of vibrations or any particular device for inducing vibrations.

As just mentioned, this application describes devices, systems and methods that help enhance breast milk pumping by vibrating the milk ducts to increase the volumetric flow rate of the milk. A typical breast pump includes a vacuum motor and a solenoid. During each pumping cycle, the vacuum motor turns on, creating pressure at the breast and thus helping express milk. At the end of the cycle, the pressure is released by turning on the solenoid to normalize the pressure in the breast pump flange. The cycle is then repeated. By “repeated,” it is meant simply that multiple cycles run in succession, for as long as the breast pump is activated. In some cases, the same cycle may be repeated over and over again—i.e., cycles with the same waveform. In other embodiments, the cycles may differ. For example, two different cycles may alternate. Or the cycle waveform may change over time. Or the cycle waveform may be adjustable or have automatic changes over time, according to a built-in algorithm. Therefore, in any given embodiment, the cycles may repeat or vary over time.

In one embodiment of breast pumps according to die present disclosure, to generate the vibratory waveform, the breast pump uses pulse width modulation on the control signal to the vacuum motor to turn the motor on and off rapidly. The vacuum motor can be driven by an h-bridge to cyclically create a vacuum and release the vacuum, by alternating the polarity to the motor. In some embodiments, the breast pump may include more than one vacuum pump. One vacuum pump provides the non-vibratory waveform, while the other vacuum pump provides the vibratory effect by increasing and/or decreasing pressure.

In another embodiment, a method for inducing a vibratory waveform in a breast pump cycle may involve modulating the solenoid while the vacuum is on. The breast pump may include more than one solenoid. One solenoid, selected to provide a fast release time, may be used to release the vacuum. The other solenoid, selected to have a slow release time, may be used to provide the vibratory waveform.

In other embodiments, the vibratory waveform may be generated mechanically by the design of the vacuum pump. For example, in a multiple n-piston-based vacuum pump, m pistons (where m<n) can be non-connected or connected to a release valve, which will create the stepwise vibratory pressure profile. In the multiple n-piston-based vacuum pump, the pistons may be aligned asymmetrically, to provide the vibratory waveform. Alternatively or additionally, valves within the piston vacuum pump may be purposely designed to be “leaky,” to provide a partial release in vacuum to create a more pronounced vibration effect. Other mechanical alterations may include designing a release valve that automatically turns on and off rapidly to create the vibration. The vibration may also be created by a motor squeezing and releasing a bulb or balloon that is in-line with the vacuum pump.

In various embodiments, vibrations may be generated on the flange or bottle assembly of the breast pump device. Mechanisms that may be incorporated into a breast pump device to generate vibrations on the flange or bottle assembly include, but are not limited to, a linear or rotary vibration motor, a piezo-electric crystal, a shape memory alloy, a speaker, and a magnet. For example, one breast pump device may include a motor positioned directly on the flange. The motor may include an offset weight attached to the motor shaft, to create vibrations in the flange, which are transmitted to the breast and ultimately to the milk ducts.

Alternatively, vibrations may be generated using an external device. Such a device may be placed or won on the breast and may create vibrations by any suitable mechanism(s).

The frequency and amplitude of the generated vibrations may be varied, in order to induce or sustain let down, make let down happen easier by lowering the sensation threshold of the body, and/or vibrate the milk to make it flow more easily by reducing the shear stress of the fluid and/or frictional coefficients of the fluid against the ducts. To conserve battery power, generated vibrations may have a low frequency and a low amplitude. Alternatively, any combination of frequency and amplitude may be used.

Any features or components described in this application for generating a vibratory waveform in a breast pump may be used with or incorporated into any suitable powered or non-powered breast pump device. The vibratory waveform may be used as a third method for controlling the pumping apparatus, in addition to (or as an alternative to) adjusting the breast pump's cycle speed and/or suction level. In various embodiments, the vibratory waveform may be tuned by the user and/or by a feedback control mechanism built into the device. The vibratory waveform may help vary the vibration level within the waveform or against the breast tissue so that the variables of suction, vacuum and vibration could be independently controlled by the user manually or by an automated or adaptive learning computer algorithm, to support the optimization of milk output.

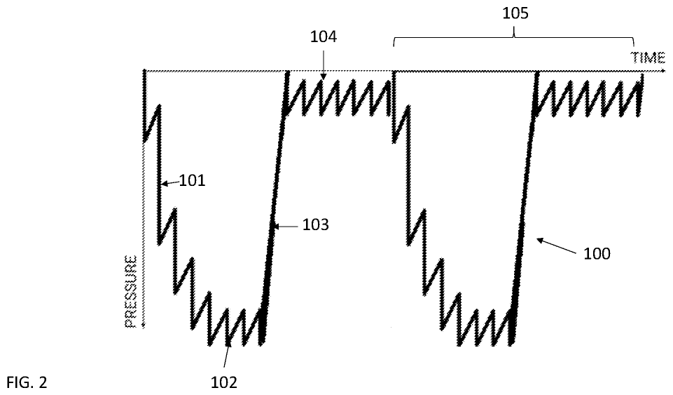

Referring now to FIGS. 2-10B, according to various examples and embodiments, many different vibratory waveform shapes, types, patterns, sizes, etc. may be generated and used in a breast pump device to enhance milk extraction from a breast. FIGS. 2-108 illustrate examples of such vibratory waveforms. Later figures depict examples of devices that may be used to generate the vibratory waveforms. In general, any vibration inducing device described herein may be used to generate vibrations having any waveform or other characteristics, unless specifically described otherwise. Thus, the scope of the present application should not be limited to the use of any specific vibration device or any specific vibratory waveform.

FIG. 2 is a time versus pressure graph that shoes one embodiment of a vibratory waveform 100, which may be generated in a breast pump using the methods and devices described herein. Each complete cycle 105 of the vibratory waveform 100 includes an increasing vacuum segment 101 (or “reduction in pressure segment”), a vacuum hold segment 102, a vacuum release segment 103 (or “normalizing the pressure segment” or “venting segment”), and a final hold segment 104 (or “normalized pressure hold segment”). In this embodiment, the vibrations of the vibratory waveform 100 are applied during the vacuum segment 101, the vacuum hold segment 102, and the final hold segment 104, but not during the vacuum release segment 103. The oscillatory effect of the normalized pressure hold segment 104 may occur at the normalized pressure, slightly higher than normalized pressure, or most preferably lower than normalized pressure—e.g., a slight vacuum, to help maintain the breast in the correct suction position within the flange of the breast pump. The waveform 100 may be repeated for any number of cycles 105, in the same pattern or a different pattern. The pattern of the waveform 100 may be changed, according to various embodiments, automatically, manually or both. For example, the pattern may be adjusted manually by the user by varying settings of the breast pump device. Alternatively or additionally, the pattern may be adjusted automatically by a control unit of the breast pump device, which may be directed via computer software through tunable or reactive learning interactions.

As mentioned above, currently available breast pump systems typically allow a user to adjust (or adjust automatically) the cycle speed and suction pressure of the system. Referring to the waveform 100 of FIG. 2, adjusting the cycle speed would change the “width” of each cycle 105 along the horizontal “time” axis of the graph. A faster cycle speed equates to higher frequency, and a lower cycle speed to lower frequency. Adjusting the suction pressure would change the “height” or “depth” of the curve along the vertical “pressure” axis of the graph. According to various embodiments described herein, the user and/or the control unit of the breast pump system may adjust vibrations in addition to or as an alternative to adjusting cycle speed and/or suction pressure. Vibration adjustments may include, for example, turning vibrations on or off, making vibrations occur over different portions of the waveform 100, and/or changing a pattern or depth/strength of each vibration. In some embodiments, for example, the breast pump system may include one or more dials, switches, buttons, sliders or the like, for making the adjustments. Some embodiments may include a separate controller, such as a remote control unit or a computer application downloaded on a smart phone, tablet, etc. Generally speaking, any given embodiment may allow a user to adjust or control vibrations, cycle speed and/or suction pressure in any suitable combination.

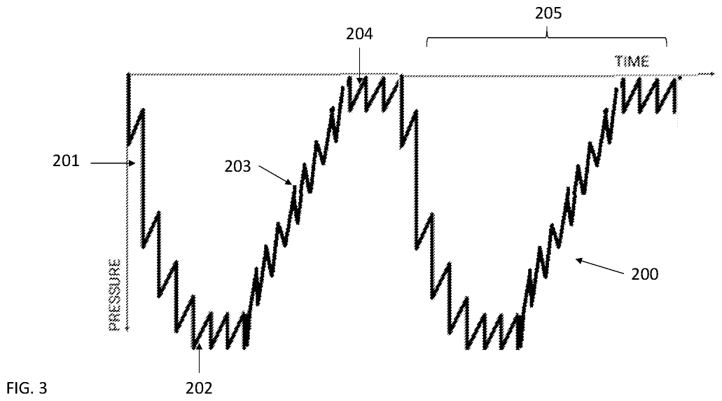

Referring now to FIG. 3, another embodiment of a vibratory waveform 200 for use with a breast pump device is illustrated. In this embodiment, the waveform 200 includes an increasing vacuum segment 201, a vacuum hold segment 202, a slow vacuum release segment 203, and a restart segment 204 at or near normalized pressure, which may contain a vibratory pattern. In this embodiment, vibrations are applied throughout the entire cycle 205 of the waveform 200, although vibrations during the restart segment 204 are optional. According to various embodiments, the segments 201, 202, 203, 204 may repeat in any configuration of these patterns or other patterns of vibration, suction, stair step, etc. The vibration patterns disclosed herein are also be interchangeable between each other, so that a user of a breast pump device may experience multiple different types of patterns within one operational period of the device.

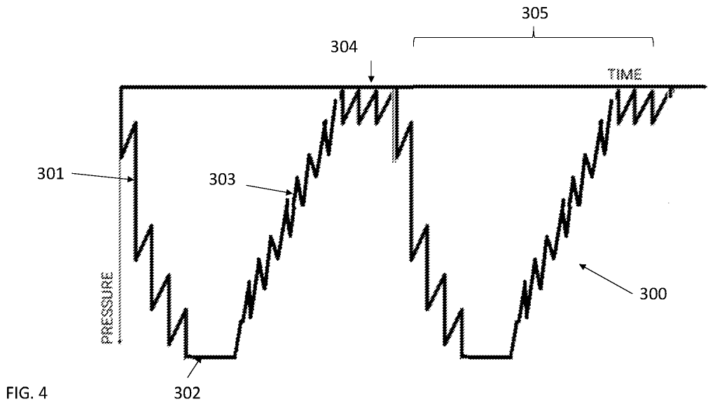

FIG. 4 shows another embodiment of a vibratory waveform 300 for use with a breast pump. In this embodiment, each cycle 305 of the waveform 300 includes an increasing vacuum segment 301, a hold vacuum segment 302, a slow vibratory vacuum release segment 303, and a near normalized pressure segment 304. In this embodiment, vibrations are applied during all segments other than the hold vacuum segment 302, which is vibration free. For this waveform 300, the normalize pressure segment 304 is optional, meaning that in some embodiments one cycle 305 may end with the vibratory vacuum release segment 303, and the next cycle may immediately begin with the increasing vacuum segment 301.

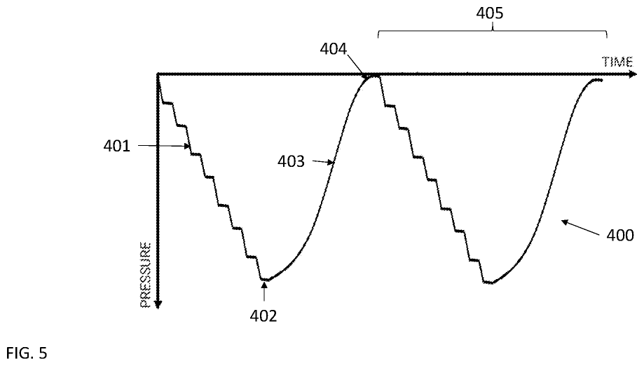

FIG. 5 depicts another embodiment of a vibratory waveform 400 for a breast pump suction profile. In this embodiment, each cycle 405 of the waveform 400 includes a vacuum segment 401, a maximum vacuum segment 402, a vacuum release segment 403, and an end cycle segment 404. The vacuum segment 401 has a stair-step pattern of vibrations applied to it. The maximum vacuum segment 402 may include a hold period, during which vacuum is maintained, but such a period is optional.

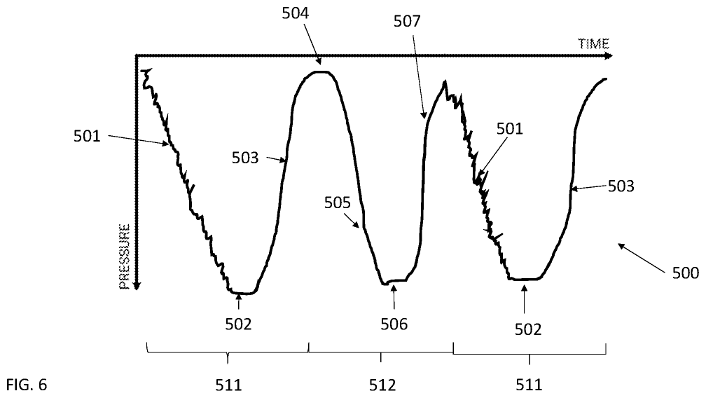

With reference now to FIG. 6, another embodiment of a vibratory waveform 500 for a breast pump is illustrated. This embodiment includes two types of waveform cycles a first cycle type 511 and a second cycle type 512. The first cycle type 511 includes an increasing vacuum segment 501 with micro-oscillation vibrations, and a hold vacuum segment 502, a vacuum release segment 503 and an end segment 504, all with no vibrations. The second cycle type 512 includes an increasing vacuum segment 505, a hold vacuum segment 506, and a vacuum release segment 507, all with no vibrations. These cycles 511, 512 of the vibratory waveform 500 may be performed in any order desired by a user. The embodiment of FIG. 6 includes two different types of cycles 511, 512 in a single waveform 500, but other embodiments may include more than two different types of cycles, different patterns of differing cycles, oscillation between two or more cycle profiles, and/or the like. In various embodiments, any of the waveform shapes, patterns, types and/or sizes described herein may be combined with any other waveform shapes, patterns, types and/or sizes, whether described herein or not, in any combination and number, without departing from the scope of this disclosure.

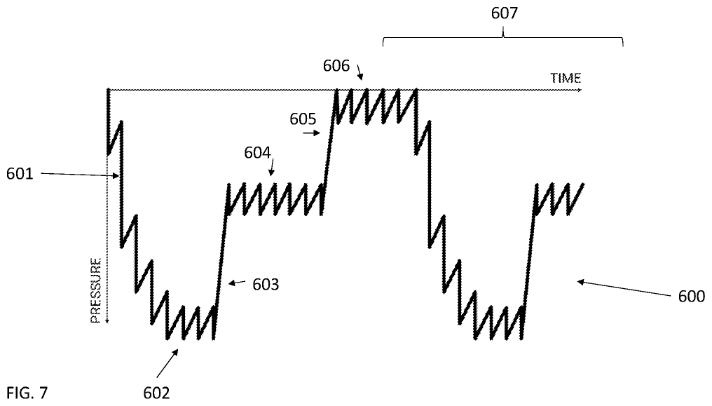

FIG. 7 depicts another embodiment of a vibratory waveform 600 for a breast pump suction curve, in which each cycle 607 includes a vacuum increase segment 601, a vacuum hold segment 602, a first vacuum release or vent segment 603, a partial reduced vacuum hold segment 604, a second vacuum release or vent segment 605, and an end of cycle segment 606, at which pressure is near ambient normal. Vibrations are applied at all segments other than the first vacuum release segment 603 and the second vacuum release segment 605. Variations on this embodiment of the waveform 600 may include different combinations of more or fewer hold segments, vacuum increase segments and/or vacuum decrease segments. Additionally, the same elongated stair-step vibration pattern used in the vacuum increase segment 601 may be applied in one or both of the vacuum release segments 603, 605, in alternative embodiments, to more slowly reduce the vacuum to one or more limits, to facilitate the stimulation of let down and/or the stimulation or production of breast milk and/or colostrum.

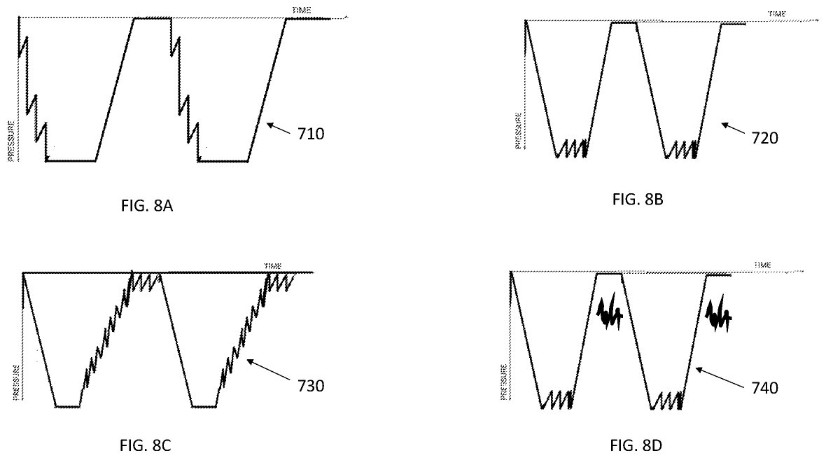

FIGS. 8A-8D show four different embodiments of breast pump suction waveform profiles with varying segments of oscillation and/or vibratory effects. FIG. 8A shows a waveform 710 with a vibration effect on the increase in vacuum side of the cycle. FIG. 8B shows a waveform 720 with a vibration effect within the maximum vacuum segment. FIG. 8C shows a waveform 730 with a vibration effect during and immediately after venting to a near normalized pressure segment. FIG. 81D shows a waveform 740 with a vibration effect upon venting to a near normalized pressure segment including increasing the pressure slightly above the current atmospheric pressure in which the pump is operating if desired. These effects may be controlled by a micro-processor within the control unit of the breast pump device (or separate from the breast pump device), which can time the effects of one or more motors and/or one or more solenoids to adjust the effect over different segments of the breast pump to produce the desired effect while pumping the breast.

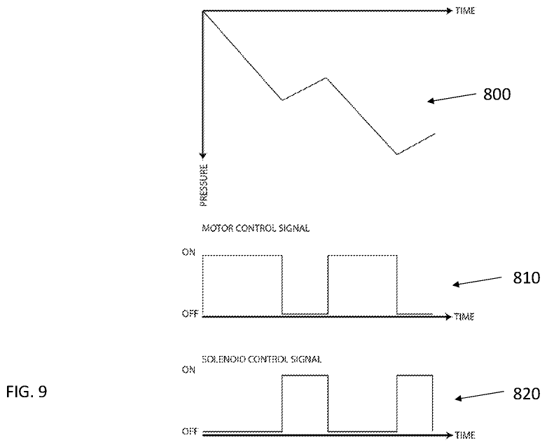

FIG. 9 includes a time versus pressure curve 800 in parallel with a motor control signal on/off curve 810 and a solenoid control signal on/off curve 820. In various embodiments, the motor and/or the solenoid of a breast pump may be tuned/adjusted by a user to produce the desired vibration and vacuum waveform 800 for pumping. This effect and/or the action of the motor(s) and/or solenoid(s) to create the vibrations and/or controlled waveform effect may additionally or alternatively be adjusted by a control unit of the breast pump, programmed with software, to facilitate specific wave forms at different times, as desired by the user and/or as informed to the control unit by sensors or feedback from the user.

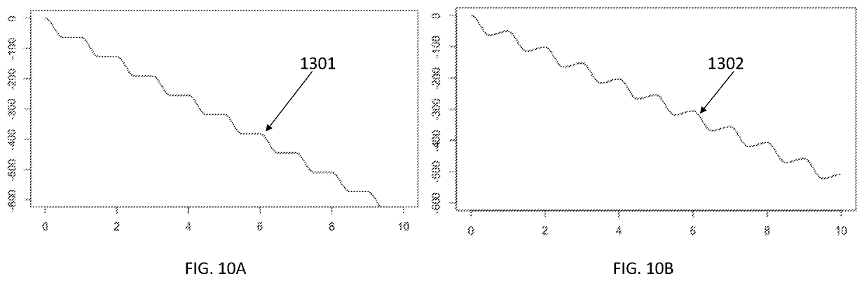

FIGS. 10A and 10B are graphs illustrating two different embodiments of vibratory waveforms. In FIG. 10A, the vibratory waveform 1301 has a stair-step pattern. One method for generating such a pattern is to rapidly turn the breast pump on and off repeatedly. This may be achieved, for example, by using a stepper motor or a DC motor. When the breast pump is on, vacuum is increased. When the pump is off, vacuum is held.

In FIG. 10B, the vibratory waveform 1302 has a wavy pattern created by repeated oscillatory increases and decreases in vacuum. One method for generating this type of wavy patterned waveform is by having a separate vacuum motor or m piston (where m≤1 and m≤n) within a n-piston vacuum motor increase and/or decrease the vacuum within the system. Another method to generate this pattern is a controlled partial release of vacuum by using a solenoid.

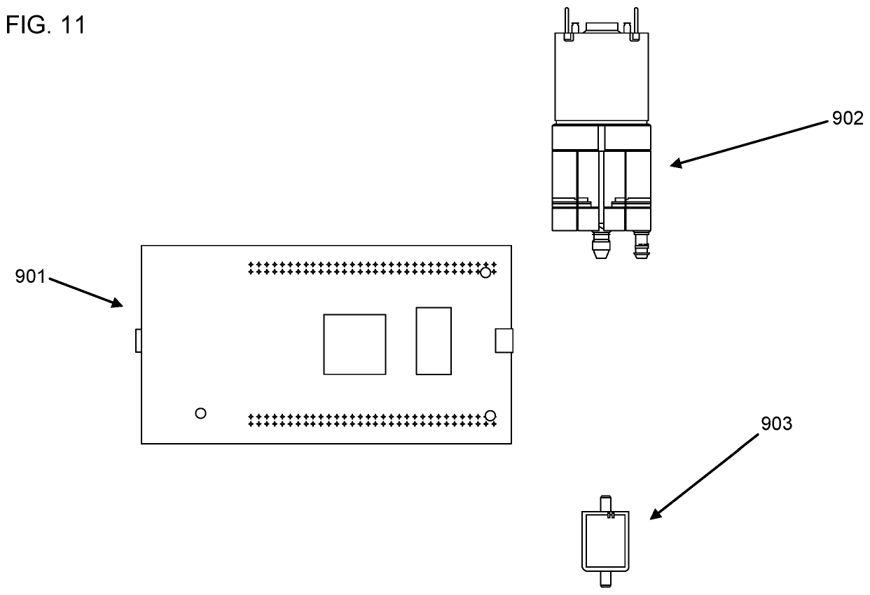

FIG. 11 illustrates three components that may be included in a breast pump device or system and that may be used, in various combinations, to provide a vibratory waveform. These components may include a printed circuit board (PCB) 901 (or other similar electronic components), a motor 902, and a solenoid 903. Various embodiments of a breast pump may include multiple PCBs 901, multiple motors 902, and/or multiple solenoids 903, and that fact will not be repeated each time any of these components is mentioned. The PCB 901 may work together with the motor 902 and/or the solenoid 903 to provide vibrations to the breast pump cycle, as described above. In alternative embodiments, other types of pressure venting devices may be passively, electrically, or mechanically actuated in combination with the pump motors, pressure regulator valves, and/or other components, to create the desired wave form within the suction induction curve.

FIG. 12 is a side view of a breast pump device 1000, according to one embodiment. This and several following figures will refer to the breast contacting portion of the breast pump system as the “breast pump device.” Not shown are the control unit (or “pump”) and the tubing for connecting the breast pump device with the control unit. As mentioned previously, the specific terminology used for various components of a breast pump system should not be interpreted as limiting.

In this embodiment, the breast pump device 1000 includes a vacuum port 1001, a pressure regulation diaphragm 1004, a collection receptacle 1003 for milk or colostrum, a vibration device 1002, and a funnel 1005 with an opening 1006 for accepting a breast. The vibration device 1002 is a small vibration inducing motor attached to a proximal portion of the funnel 1005. In alternative embodiments, the vibration device 1002 may be attached to a different part of the breast pump device 1000, such as but not limited to a flange, the collection receptacle 1003 or the diaphragm 1004. In the pictured embodiment, the vibration device 1002 directly vibrates the funnel 1005, which conducts the vibrations into the breast tissue received in the opening 1006. The vibration device 1002 may generate any of the various types and patterns of vibratory waveforms described above or any other suitable vibrations.

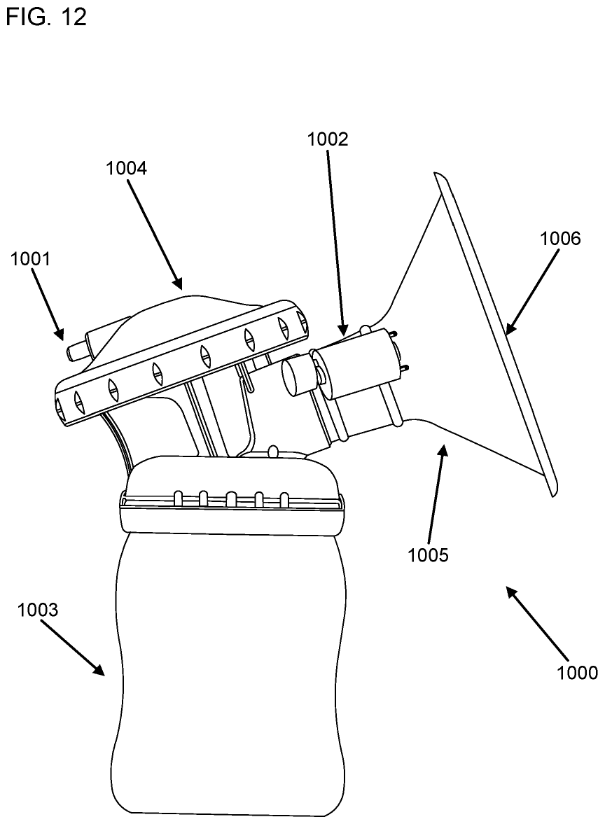

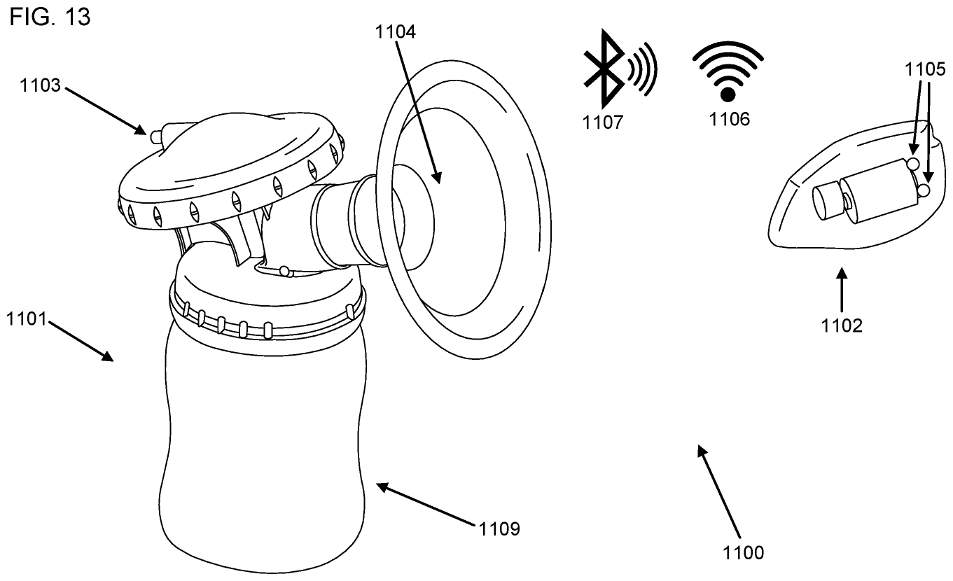

Referring now to FIG. 13, in another embodiment, a breast pump system 1100 may include a breast pump device 1101 and a separate vibration device 1102. Again, the source of suction—i.e., the breast pump housing mechanism with the motor(s), power cord, etc.—is not shown, but it may be included as part of the system 1100. The breast pump device 1101 includes a vacuum port 1103, a funnel 1104 and a collection receptacle 1109, among other parts. The separate vibration device 1102 may include a small motor for creating vibrations, and it may be held by the user against the breast or attached (e.g., adhesive) temporarily to the breast. The vibration device 1102 may include one or more signal transmitters 1105, receivers and/or transceivers, which communicate with a breast pump control unit (not shown) through wired or wireless connections, such as WIFI 1106 and/or Bluetooth 1107. Although not required, this communication could, in combination with sensors in the vibration device 1102 and/or the breast pump device 1101, provide feedback for the microcontroller to adjust the actuation of the pressure in the breast pump waveform and/or the level of vibration produced by the vibration device 1102. This feedback loop may be preset into the breast pump system 1100 in some embodiments.

FIG. 14 is a side view of a breast pump device 1200 according to another embodiment. In this embodiment, the device 1200 includes all the features of a typical breast pump device, such as a collection receptacle 1203, a funnel 1205, a suction port 1207, etc. In addition, the device 1200 includes an eccentric motor 1202 attached to the top or lid portion of the collection receptacle 1203. The eccentric motor 1202 generates vibrations, which vibrate a membrane 1201 disposed in the funnel 1205, thus resulting in an oscillatory increase and decrease of vacuum (vibration) in the vacuum waveform. The eccentric motor 1202 may communicate to the breast pump control unit through wireless or wired technologies. The eccentric motor 1202 may be attached as part of the breast pump device 1200 or may be a separate piece that can be attached by the user, according to various embodiments.

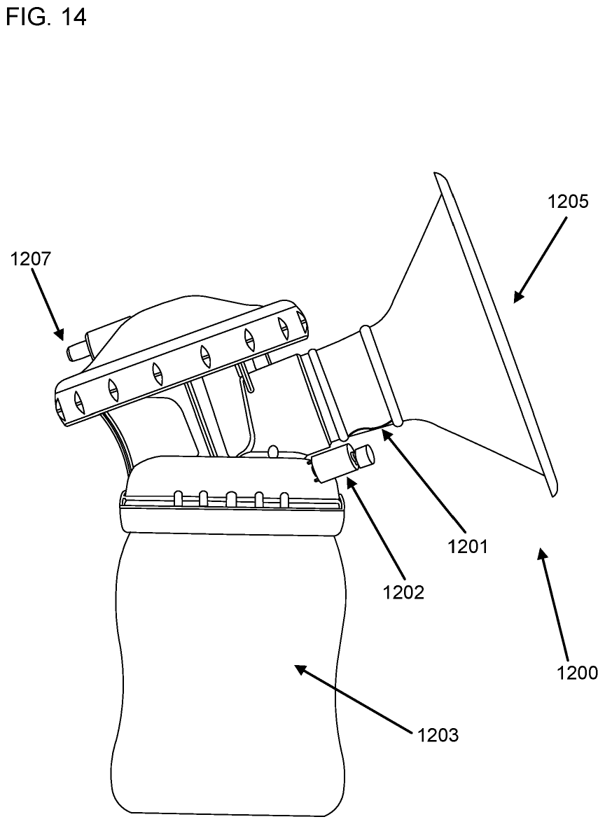

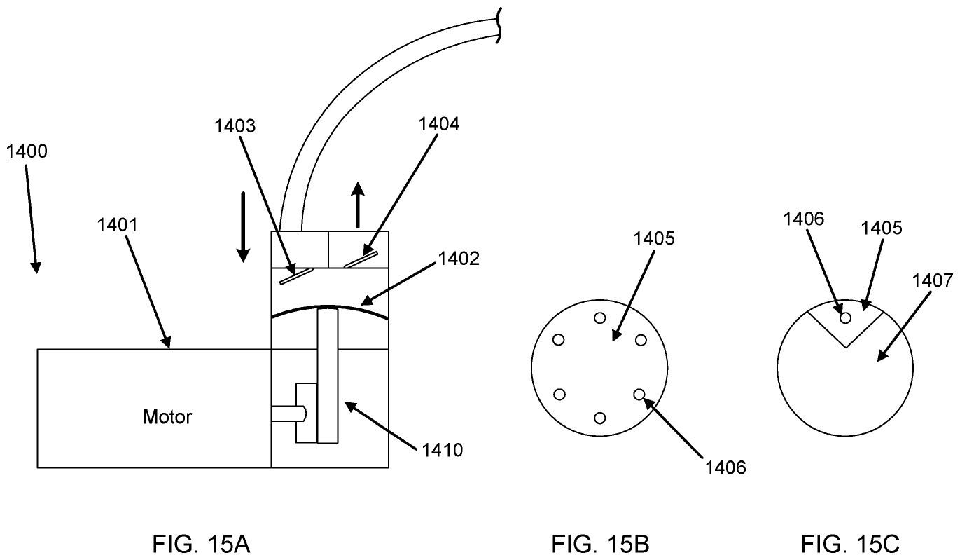

Referring now to FIG. 15A, one embodiment of a vacuum motor device 1400 for a breast pump system is illustrated. In this embodiment, the vacuum motor device 1400 includes a DC motor 1401 connected to a shaft that moves a piston 1410 connected to a diaphragm 1402. On its down cycle, the piston 1410 pulls the diaphragm 1402 down and thus pulls air from the flange connected to the breast through a first one-way valve 1403, creating a vacuum on the breast. On its up cycle, the piston 1410 pushes the air through a second one-way valve 1404 to the outside world, thus completing the breast pump cycle. In an n=1 n-piston breast pump system, as illustrated by the device 1400 of FIG. 15A, this will produce a stair-step vibratory waveform 1301, such as the one illustrated in FIG. 10A.

Referring now to FIGS. 15B and 15C, to create an oscillatory waveform such as the waveform 1302 in FIG. 10B, some vacuum force must be released from the vacuum motor device 1400. One way to accomplish this is to pass air in the opposite direction through the first one-way valve 1403. In one embodiment, the first one-way valve 1403 may include a diaphragm 1405, as illustrated in top view in FIG. 15B. The diaphragm 1405 includes multiple holes 1406 or apertures, which allow air to pass through. (Any suitable number of holes 1406 may be included.) As illustrated in FIG. 15C, the flap 1047 of the first one-way valve 1403 may include a cut-out portion or other form of opening, to expose pan of the diaphragm 1405 and one or more of the holes 1406, which still allow air to pass in the opposite direction through the valve 1403. Air flowing through the first one-way valve 1403 in the opposite direction will cause the oscillatory waveform, because during the up cycle of the piston 1410, some of air is returned to the flange, resulting in a slight decrease in vacuum. This modification of the first one-way valve 1403 can be extended to n>1 in a n-piston vacuum motor.

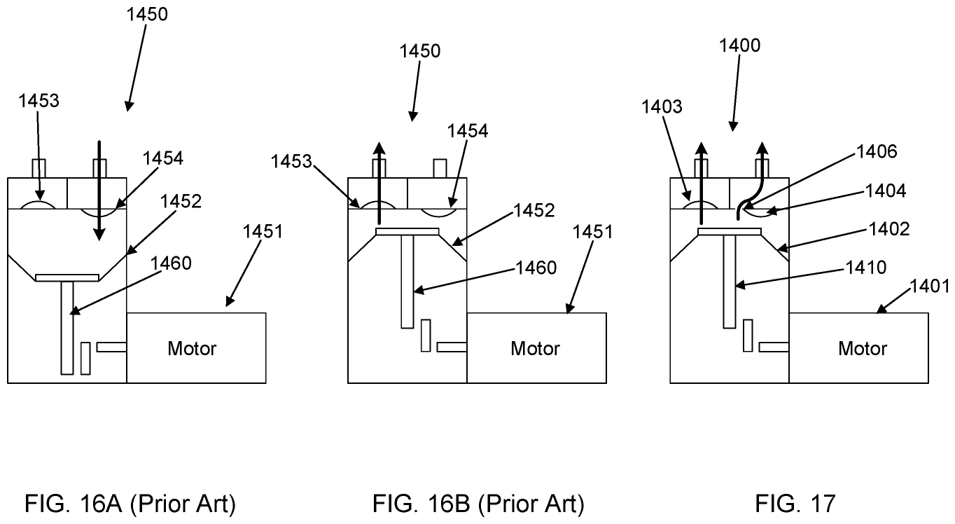

With reference now to FIGS. 16A and 16B, operation of a prior art vacuum motor device 1450 of a breast pump system is illustrated. As illustrated in FIG. 16A, the motor 1451 of the device 1450 drives a piston 1460 to pull down on a diaphragm 1452, which pulls air (down arrow) into the device 1450 through a first one-way valve 1454. This movement of air creates a vacuum force in the breast contacting portion of the breast pump system. In 16B, the motor 1451 then drives the piston 1464 upwards, pushing the diaphragm 1452 up and pushing air (up arrow) out of the device 1450 through a second one-way valve 1453. This pushed-out air releases the vacuum force from the breast contacting portion of the system.

FIG. 17 illustrates operation of the same vacuum motor device 1400 of FIGS. 15A-15C, in contrast to the prior art device 1450. In the FIG. 17 device 1400, when the motor 1401 drives the piston 1310 up to push the diaphragm 1402 up, air is pushed out of the device 1400 through the second one-way valve 1403 (thick up arrow) and is also pushed out through the hole 1406 (or multiple holes) in the diaphragm of the first one-way valve 1404 (thin up arrow). The air escaping through the hole(s) 1406 causes the vibrations in the system. In alternative embodiments, one or more holes may be placed in a part of a breast pump other than the diaphragm, such as in part of the plastic assembly.

With reference now to FIG. 18, in an alternative embodiment, a breast pump system 1500 may include a first vacuum motor 1501, a second vacuum motor 1502, a solenoid and a flange assembly 1503, all connected by a tube 1506 or other suitable connector. The first vacuum motor 1501 provides the main source of vacuum for driving the breast pump system 1500 and providing suction to the flange assembly 1504. The second vacuum motor 1502 generates the vibrations for the vibratory waveform and may be connected to the system 1500 so that the input port and the output port of the second vacuum motor 1502 are connected to the closed system 1501. For example, in an embodiment in which the second vacuum motor is an n=1 piston vacuum motor, the motor 1502 pulls a vacuum during the first phase and releases captured air during the second phase. Since the released air goes back into the closed system 1500, air will cause vibrations in the flange assembly 1504, thus providing the vibratory waveform, such as the waveform 1302 shown in FIG. 10B. In an alternative embodiment, the user may simply connect the input port, which will generate a stair-step curve.

Referring to FIG. 19, another embodiment of a breast pump system 1600 is illustrated. This embodiment includes a vacuum motor 1601, a flexible bulb 1602, an external motor 1603, a solenoid 1604 and a flange assembly 1605. The vacuum motor 1601 provides the main source of vacuum for driving the breast pump system 1600 and providing suction to the flange assembly 1605. The external motor 1603 is attached to the flexible bulb 1602 (rubber bulb or similar material), and the two work together to generate the vibratory waveform. First, the external motor squeezes the bulb 1602 to expel air into the system 1600. The expelled air decreases the overall vacuum in the flange assembly 1605. When the external motor 1603 relaxes and allows the bulb 1602 to expand, air is pulled back into the valve, thus increasing the overall vacuum in the system 1600. Thus, the vibratory waveform is provided.

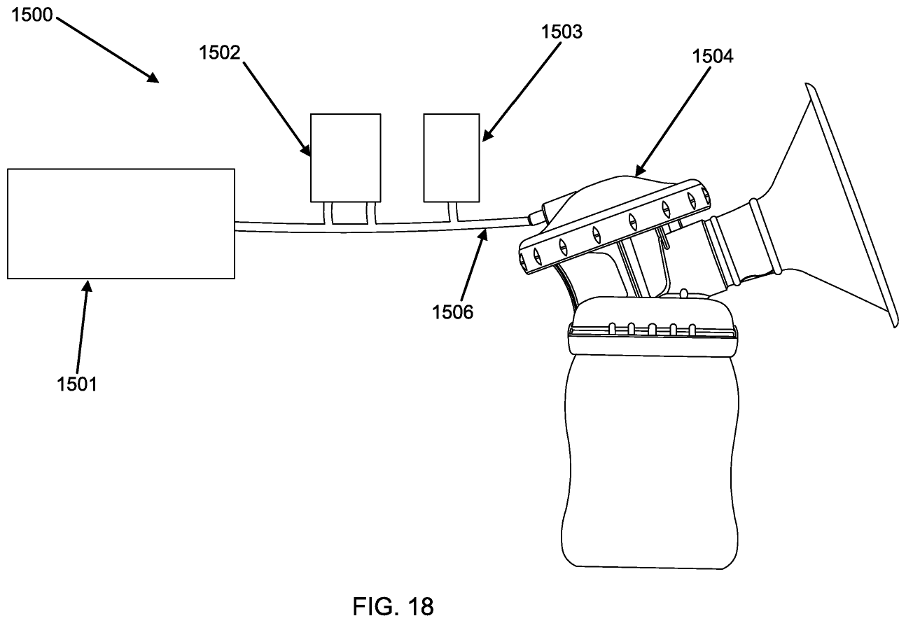

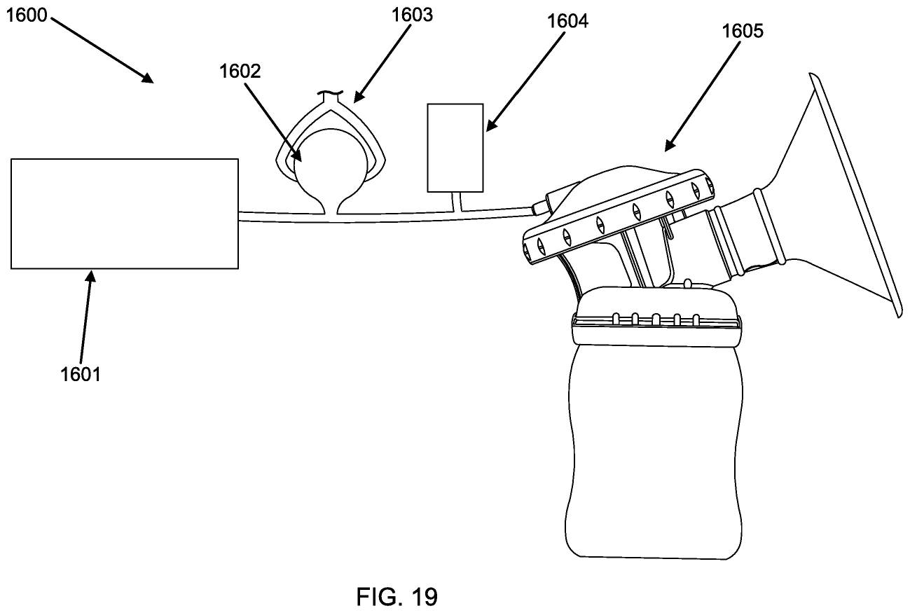

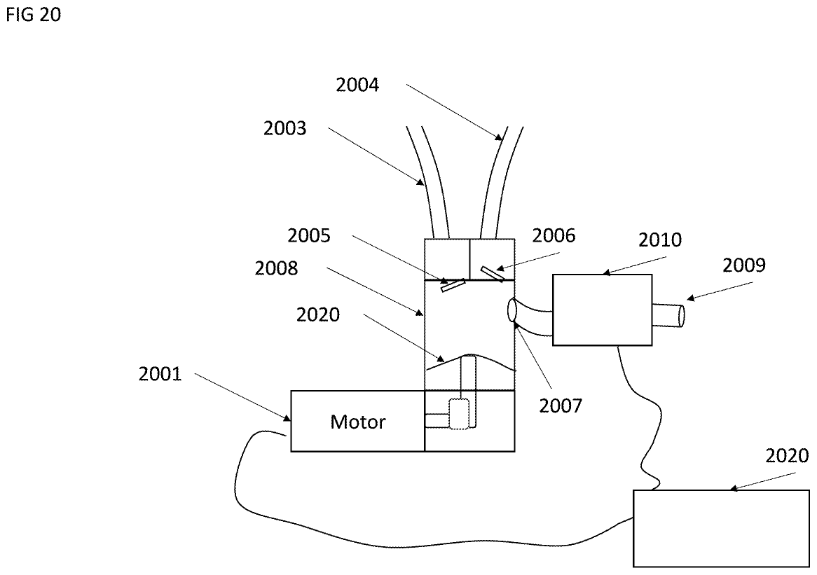

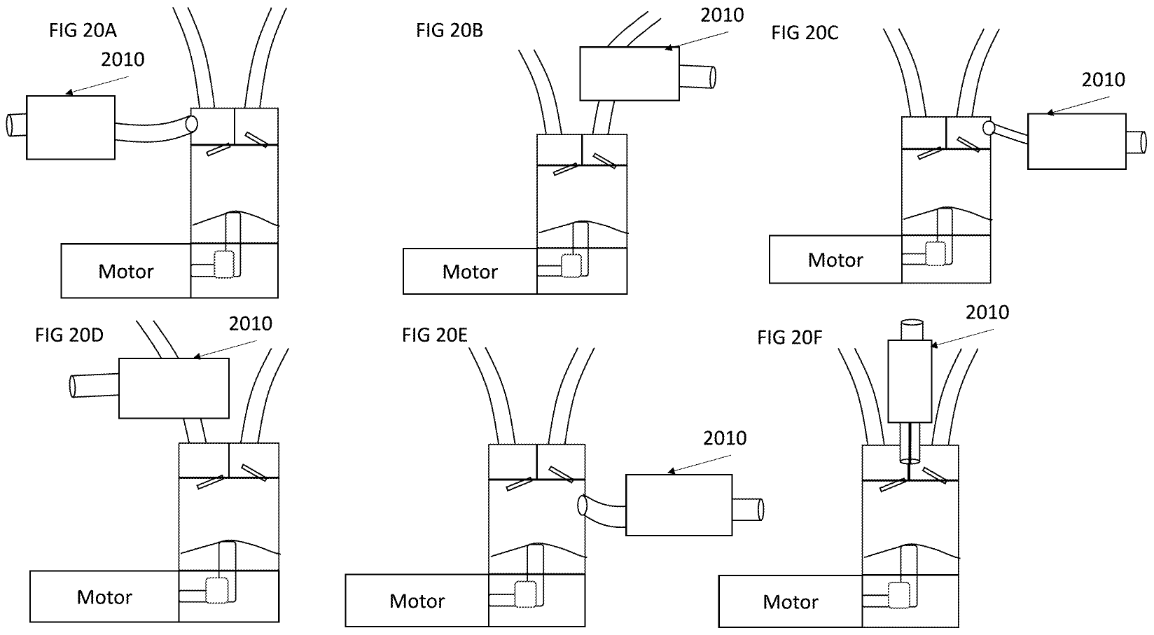

Referring now to FIGS. 20-26, alternative embodiments for systems and methods of introducing a vibratory waveform into a pumping system are shown. Generally, as described previously, these embodiments allow for the introduction of a continuous and/or a non-continuous leak of one or more magnitudes of variation over time that causes the desired vibration at the desired time(s) throughout the vibratory waveform. In such examples, the user (e.g., the woman who is pumping milk from her breasts) of the pumping system can control various aspects of the vibration, including one or more of timing, duration, and intensity.

As noted above, introducing vibration into the waveform of a breast pump cycle may enable some women to get let down faster, more efficiently express milk, and/or achieve breast pumping at a higher level of suction with increased comfort. However, other women may notice no benefit or even small decreases in expression and/or let down due to the added vibration. For these women, it may be desirable to allow them to decrease the frequency or magnitude of the vibration or to cease the vibration.

In the embodiments described below, the vibration can be generated by disrupting the flow of the air entering or exiting the breast pump vacuum circuit through several always-on and/or reversible embodiments. Always-on embodiments would enable the system to always contain some level of vibration within the system. There am multiple embodiments described herein that could be configured to be always on due to the fundamental design of the embodiment. Alternative embodiments could be reversible or configured in such a way that the alternate embodiments are always on.

Alternatively and/or in addition, there could be reversible embodiments that allow for the vibration to be turned on or off partially or completely or alternatively as embodiments for introducing or modulating variation in the vibration amplitude and frequency by manipulating the operating parameters of the vibration mechanism and/or the pump system. For instance, the operating parameters of one or both of vacuum motor(s) and electromechanical switch valve(s) of the vacuum motor devices described below can be controlled by the user to accomplish a desired manipulation of the vibrations by adjusting the amount of air entering or exiting the system. Each of these embodiments can provide unique benefits to the women who operate the pump in order to enable more efficient and optimized pumping based on the preferences of the women using the pumps.

Referring now to FIG. 20, another embodiment of a vacuum motor device 2000 for a breast pump system is illustrated. In this example, the vacuum motor device 2000 uses an electromechanical device 2010, such as a solenoid or switch, to oscillate and/or purposefully leak air into the system from the outside during the system operation. This can be accomplished with a normally-open electromechanical device or a normally-closed electromechanical device.

In general, a vacuum motor 2001 would be operating to create vacuum through the action of a diaphragm 2002, and the electromechanical device 2010 would be opened and closed or oscillated multiple times during the vacuum increase phase in order to generate a vibratory disruption in the vacuum increase phase of the natural operation of the vacuum motor 2001. This same effect could be achieved on the vacuum release phase of the electromechanical device release to generate vacuum vibrations as the vacuum is releasing as well as at any stage of the hold at the low level or atmospheric level of vacuum or any level of hold in between a minimum and maximum level.

Other components of the system may be configured in many different manners, as evident in FIGS. 20A, 20B, 20C, 20D, 20E, and 20F. In each embodiment, as in FIG. 20, there may be additional components that enable the motor system (2011) to have air suction intake (2003) and outlet (2004) through a system of inlet valve or valves (2005) and outlet valve or valves (2006). The electromechanical device (2010) has a communication conduit (2007) or opening with the motor housing (2008) such that it can allow for air to enter through the communication conduit (2007) when the electromechanical device (2010) is configured to allow for its internal valve system to open a port to allow air to come into the system from an air inlet (2009) attached to the electromechanical device (2010).

In some embodiments, the entire system may be fully integrated such that the communication conduit (2007) may not be required if the electromechanical device (2010) is directly connected to the motor system (2008). Additionally, in other embodiments, an electromechanical mechanism may not be needed, and the device described as an electromechanical device (2010) could be configured to be a pressure relief valve or spring system configured to actuate trader pre-defined pressure targets from springs or other tensions or mechanical-only components made to operate at specified cracking pressure(s) such as, but not limited to, a flow restrictor valve or pressure relief valve system.

This functionality can be turned on or off by the user through the use of a control mechanism 2020, such that the timing and frequency of oscillation by the electromechanical device 2010 could create different amplitudes and/or other operating parameters of the vibration while also giving the user ability to turn the vibration function off completely. The control mechanism (2020) could comprise a complex of a PCBA—with or without: power, voltage, current and/or pressure sensors or other sensors, and/or power circuits with AC or DC power including wall connected power and/or battery power.

This tenability could also be adjusted by the firmware of the system such that it compensates for differences in the vacuum release speed at different vacuum levels, to maintain a constant vibration amplitude of the vibration regardless of the vacuum level. For example, at low vacuum, it might require 0.1 seconds to release 10 mmHg, while at high vacuum, it might only require 0.05 second to release 10 mmHg. In other embodiments, it may be desired to anti-compensate or not-compensate for this different vacuum release speed or rush in air speed during the vibrations being created such that vibration amplitudes could differ along the suction curve of decreasing vacuum.

The vacuum motor 2001 could also be tuned such that it could operate more strongly or less strongly in conjunction with the electromechanical device 2010 to control the vibration rate of vacuum release during the decreasing or increasing vacuum phase or hold phase in order to have the motor operation provide additional control over the desired performance characteristics of the system. For example, if the timing resolution of the solenoid is limited to 100 ms, at high vacuum it might have released too much vacuum, for example 20 mmHg instead of the desired 10 mmHg. To compensate, the vacuum motor is driven on at higher power for the additional vacuum loss, resulting in the desired 10 mmHg amplitude.

Using a combination of operating parameters associated with the vacuum motor 2001 and the electromechanical device 2010, the user (and possible the vacuum motor device 2000 itself) could manipulate the motor device 2000 to provide for a wide variety of unique waveforms of vacuum phases and vibrations, including turning off the vibrations completely.

In another embodiment of the vacuum motor device 2000, the amplitude of the vibration can be increased by determining the natural resonance frequency of the system and modulating (e.g., turning on and off) the electromechanical device 2010 at the same resonance frequency. The constructive interference of the pressure wave will result in a noticeable sensation on the breast. Similarly, if the user desires to rapidly remove or dampen the vibration completely, then the same resonance frequency could be used as a method of turning on and off the electromechanical device at the right time to result in deconstructive interference of the pressure wave to create a smooth or smoother wave variation for the end user.

In this fashion, the user could tune the strength of the magnitude of the vibration based on personal preference including to smooth the vibration or increase in the strength of the vibration or changing it throughout a pumping session to increase and decrease over time. This controlled mechanism could also be done by a computer system or processing algorithm to optimize user preferences and/or production over time through a closed or partially closed loop with or without user directed input. In addition, the vacuum motor device 2000 could use algorithms on the pump or in the cloud or a mobile device linked by Bluetooth or other method to automatically learn the best frequency and method of operation that would result in a mom's comfort and/or efficient expression of breastmilk.

For example, the vacuum motor device 2000 could store certain operating parameters locally or on a profile remotely (e.g., in the cloud) relating to the user's preferences, such as the vibration frequency, amplitude of the vibration, if vibration is present or should not be present during stimulation and/or expression modes or none of the modes, random vibration patterns or consistent patterns, and any other variations in combination thereof. These user preferences could be displayed on a mobile device in an application or system if linked via a connection, Bluetooth, radio frequency, Wi-Fi, or other data transmission system that would provide input and data from one device to another.

This display could additionally be interacted with by the user to adjust such parameters on a mobile device and enable the data characteristics to be sent to the pump system such that it could operate according to the inputs provided with or without any option to input such parameters on the display of the pump system hardware unit. Upon a subsequent use of the vacuum motor device 2000 (or even a different device with data connection to the user's secondary device system), these preferences can be automatically applied so that the device performs as desired by the user.

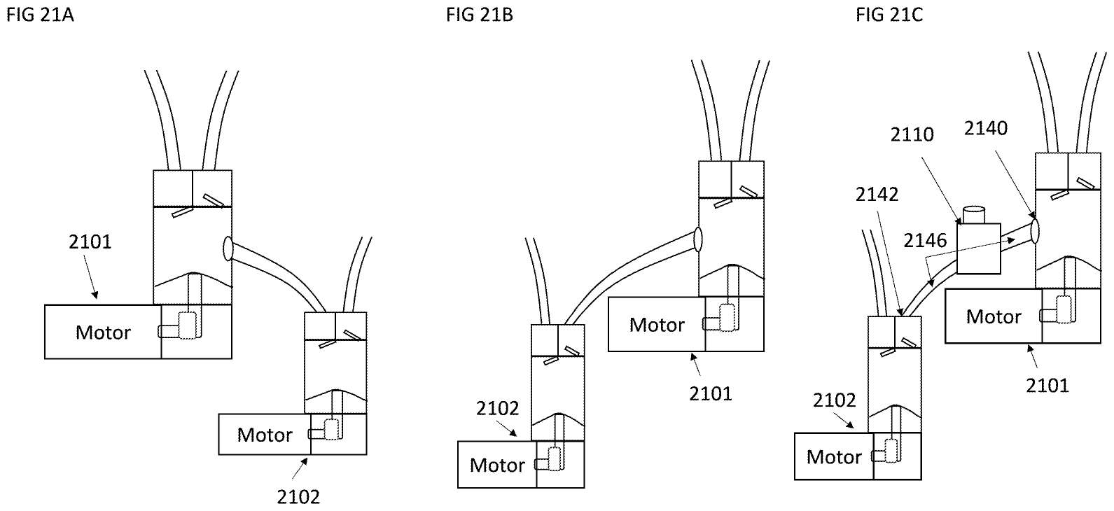

Referring now to FIGS. 21A and 21B, two additional embodiments are shown of a vacuum motor device 2100 for a breast pump system. In this example, a secondary vacuum motor 2102 is run in reverse (A) or in cooperation (B) with respect to a primary vacuum motor 2101 at periodic oscillating frequencies. This allows the secondary vacuum motor 2102 to inject air into or extract air from the system as it is being removed during an increasing vacuum wave from the primary vacuum motor 2101 on one or more oscillation cycles of one secondary motor (2102) in or out of phase with the primary motor (2101).

Similarly, variability could be introduced by pairwise or anti-pairwise operation of the two motors 2101, 2102 (or possibly more motors) at any point in the vacuum wave such as a decrease, hold, increase, or atmospheric hold to generate the desired effects. A variety of configurations and connections could be made by one or more additional motors or electromechanical switches in the system, such as but not limited to using the variety of the configurations shown in FIGS. 20-20F, with such additional motor connections to the variety of different chamber connection points shown with or without the secondary or primary motor connected in a series or a parallel circuit from the connection points. If connected in a series circuit, the air would flow from the primary motor (2101) through the secondary motor (2102), instead of the parallel bifurcated connections supplementing vacuum or air input or output us described in FIGS. 21A and 21B.

In addition, FIG. 21C illustrates an embodiment in which pressure can be built up by the secondary vacuum motor 2102 in a secondary circuit 2142 or in a primary circuit 2140 to infuse into the system (under the action of an electromechanical device 2110, which opens and closes a connection pathway 2146) this higher pressure at a desired time to the main or secondary circuits 2140, 2142 of vacuum. This creates tuned amplitude oscillations in the system based on a set of desired parameters. Other motor or drive systems could be used in addition to a rotary piston motor. Other drive systems that could be used would include, but not be limited to, a voice coil actuator, DC Shunt Motor, Separately Excited Motor, DC Series Motor, PMDC Motor, Piezo motor, DC Compound Motor, AC motor, Synchronous Motor, Induction Motor, Stepper Motor, and many other types of systems such that they could conduct air in or out of a system.

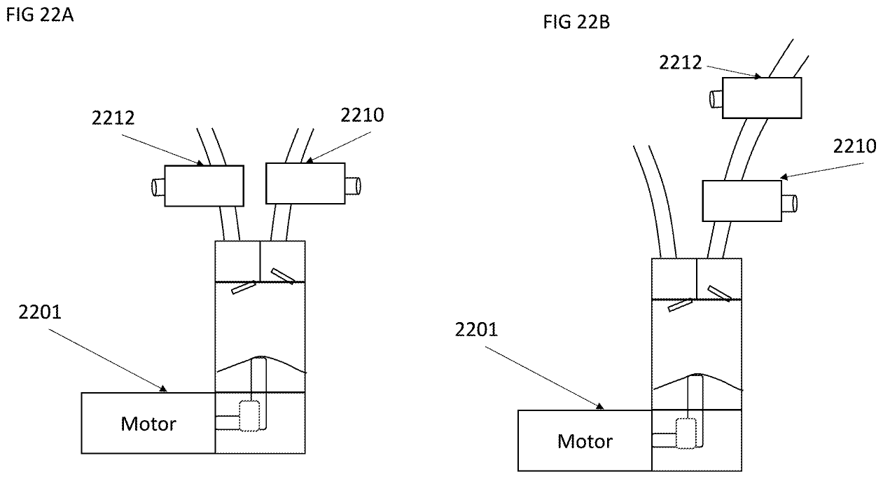

Referring now to FIGS. 22A and 22B, in further alternative embodiments of a vacuum motor system, any combination of one or more of electromechanical devices and/or one or more motors could be used in combination to generate variability in the vibratory waveform. In this depicted example, two electromechanical devices 2210, 2212 are paired with one vacuum motor 2201. The electromechanical device 2210 and/or 2012 introduces micro-vibration through its own oscillation, and the electromechanical device 2212 and/or 2010 operates only when the system prompts the full admission of air back into the system at the end of one vacuum phase cycle. In this way, the functional performance of each of the electromechanical devices 2210, 2212 (weak rapid cycle oscillation vs. slow strong open) could be separately tuned based upon functionality (introduce vibration or open the vacuum system).

In another alternative, the electromechanical device 2210 operates independently to rapidly turn on and off through one waveform creating vibration and then at the end of the waveform is held open for a period of time to fully release the vacuum. The other electromechanical device 2212 does the same, but both electromechanical devices 2210, 2212 operate independently (or at the same time). Repeatedly turning the electromechanical device on and off rapidly may degrade the operating life of the electromechanical device, therefore if operating independently, this will increase the operating life of the vacuum motor device by creating part redundancy.

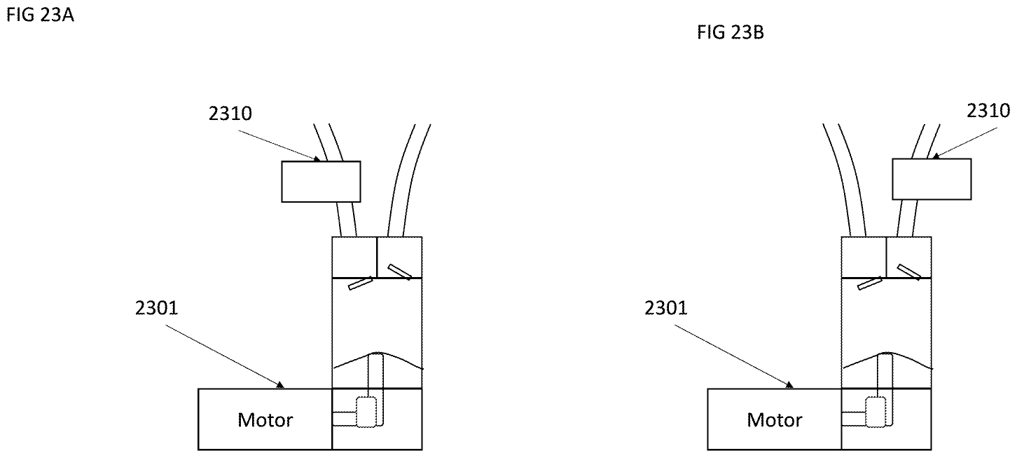

Referring now to FIGS. 23A and 23B, other alternative embodiments of a vacuum motor breast pump device may include an electromechanical device 2310 positioned in series with the vacuum motor to block the vacuum suction inlet (A) or outlet (B) created by a vacuum motor 2301.

In this vacuum motor device breast pump 2300, the velocity of the air rushing through the system creates vibration. During the vacuum phase, air is removed from the vacuum motor pump via the operation of the vacuum motor 2301. As the air molecules are pulled out of the closed system of the vacuum motor device breast pump, they will obtain kinetic energy in the form of momentum.

With the electromechanical device 2310 introduced in series, the electromechanical device 2310 can block the air flow, which will disrupt the momentum on the flange side of the breast pump. This sudden change of momentum will result in a pressure wave, that will travel from the electromechanical device 2310 back to the breast. When the pressure wave hits the breast, this effect can be felt directly on the breast.

This can be optionally accomplished with a three-way solenoid for the electromechanical device 2310 to both create the vibration based on in-line suction block and also vent the system afterwards. The position of the electromechanical device 2310 could be tuned within the electromechanical device complex to achieve this.

On the vacuum motor 2301 side for this embodiment, the vacuum motor 2301 will see a sudden increase in vacuum as the volume that it sees has been greatly diminished. The vacuum motor 2301 will continue to remove the remaining air within this now reduced volume, resulting in even higher vacuum. As the electromechanical device 2310 is opened allowing the air to flow/equalize between the two sides, this will result in a second pressure wave, as the two sides have different pressure. When the second pressure wave travels to the breast, the breast will feel this effect.

Repeating this effect over and over again during the vacuum phase will result in a vibration sensation on the breast. The benefit of this configuration is that no vacuum is wasted, where the previous ideas introduced a leak into the system, which reduced the overall efficiency of the system. The amplitude of the vibration is determined by the velocity of the air. Therefore, to increase the amplitude of the vibration, the speed of the vacuum motor 2301 is increased. One key benefit of such a system incorporating oscillating block and opening points in series through an electromechanical device (2310) is that it would minimize vacuum loss and increase efficiency as compared to a configuration based on a leak or air injection into the system design.

Alternatively, multiple electromechanical devices, can be provided, where one electromechanical device is in line with the vacuum motor and another electromechanical device is on a T connector line to allow for the air to rush in from the outside during the vacuum release phase of the cycle. It should be noted that any form of oscillating electrical or mechanical switch or valve could be used in place of an electromechanical device to introduce the vibrations. In one embodiment, even a manual actuation could be achieved with a pinch point in the system from manual actuation.

In another example embodiment of a vacuum motor device, a vacuum tube is externally pinched in order to compress and retract it using a motor. This motor pinches the vacuum tube in one or more of a variety of ways, including a rotary piston that has a notch in it or a peg system in it or other peristaltic type pump with a wheel that could spin and pinch at particular patterns. This peristaltic action could be enabled through manual depression by a user or manual rotation by a user or it could be done by the motor.

In an alternative motor design when a vacuum motor operates, movement of a piston in the vacuum motor flexes and moves around inside a pump motor housing more easily as the connection to the diaphragm is made with an oscillating spring that stretches and moves as the piston rotates in the system.

In an additional embodiment, a magnetic valve restrictor or spring valve restrictor is made to push open and allow for a vacuum pump to operate such that a suction is generated. Once a breaking threshold is reached, the magnetic valve restrictor or spring valve restrictor repulsion enables to allow for air to rush in and the spring force or magnetic repulsion force to oscillate the restrictor such that a vibration is introduced. This can be done with a two-way opening or with a precisely tuned parameter placing the valve restrictor at the appropriate distance from the opening relative to the repulsion force needed such that when passed a critical point a bypass conduit would be opened to allow for air to leak around the restriction and introduce variability within the vacuum motor device.

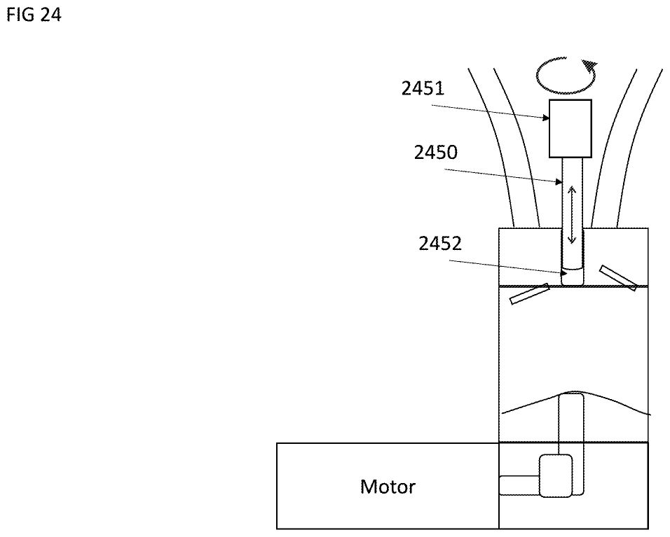

Another alternative embodiment of a vacuum motor device shown in FIG. 24 includes a screw 2450 that can be mechanically adjusted (threaded) into or out of a hole 2452 by action of an electronic rotating actuator (2451) or manual dial controlled by a user (2451). This threading of the screw 2450 within the hole 2452 opens or closes the variation in a diameter 2452 of the hole 2452. This decreases the leaking of air as the screw 2450 is threaded into the hole 2452 to thereby decrease vibration. With the screw 2450 fully seated within the hole 2452, no leaking of air is possible, thereby ceasing the vibration. Conversely, this increases the leading of air as the screw 2450 is threaded out of the hole 2452 to thereby increase vibration. The screw 2450 can be actuated by the user manually or through an electromechanical controller 2460 to thread and unthread the screw 2450 within the hole 2452.

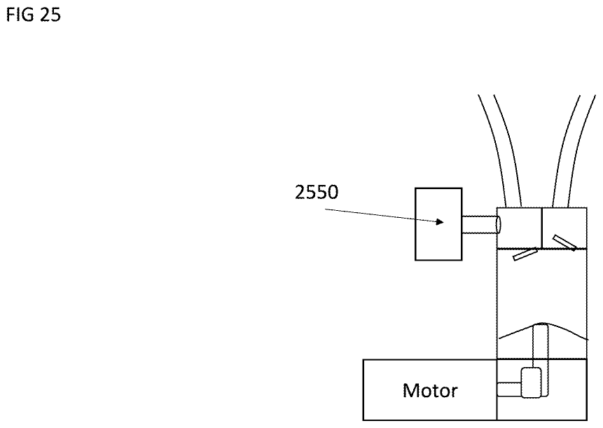

Referring now to FIG. 25, in another example embodiment of a vacuum motor device is provided with an optional bleeder or cracking pressure valve 2550 that only allows for air to leak into the vacuum motor device at a pre-specified pressure differential from the external environment.

For example, during the initial increasing vacuum phase, the valve 2550 could be open, but then as more suction is applied, the valve 2550 would close at vacuum reached above a certain threshold. For example, vibration in the vacuum phase could turn off at 180 mmHg such that the vibration could be helpful during the let down phase which typically occurs at lower vacuum levels but not at higher levels of vacuum. This could be important as higher levels of vacuum are harder to obtain if there is a leaky hole within the system.

In addition, multiple bleeder valves or alternatively one bleeder valve configured with multiple cracking pressures could be configured to let air in at different parts of the waveform, including but not limited to a beginning portion, a middle portion, and/or an end portion, such that at any section or sections of the vibratory waveform could turn on or off automatically based on what section of the suction curve is present.

For example, the vibration could not be present with the valve 2550 being closed during the initial part of the suction curve but then, after reaching a low enough pressure, the valve 2550 opens, which allows for vibration to start during the lower levels of vacuum. This could be important because higher levels of vacuum can sometimes be associated with higher levels of pain. One possible way to mitigate such pain would be to vibrate the area experiencing pain during this period of high vacuum.

For example, the vacuum increasing past 200 mmHg would result in vibration starting, whereas suction would not turn on prior to that point because the valve 2550 would be closed. Similarly, leaking could occur in lower levels to create vibration below 150 mmHg and then turn off until the vacuum motor device 2500 reaches 230 mmHg and then vibration turns back on for the remainder of the cycle as additional air leaks into the vacuum motor device 2500 from the cracked bleeder valves.

Many other types of pressure releasing valves such as a pressure relief valve or other types of spring or other types of actuated systems could be configured to result in opening and closing leaking at pre-described operating parameters in order to enable the performance desired within the specific section of the waveform or pump operation, such as, but not limited to, let down mode or expression modes.

In some variations of the prior embodiments timing of a single valve or multiple valves used in closing off could be offset to allow for two different periods of when the valve would be opened or closed. This could result in an uptick from temporary leak by making one valve stem longer than the other for example if using two pressure release valves.

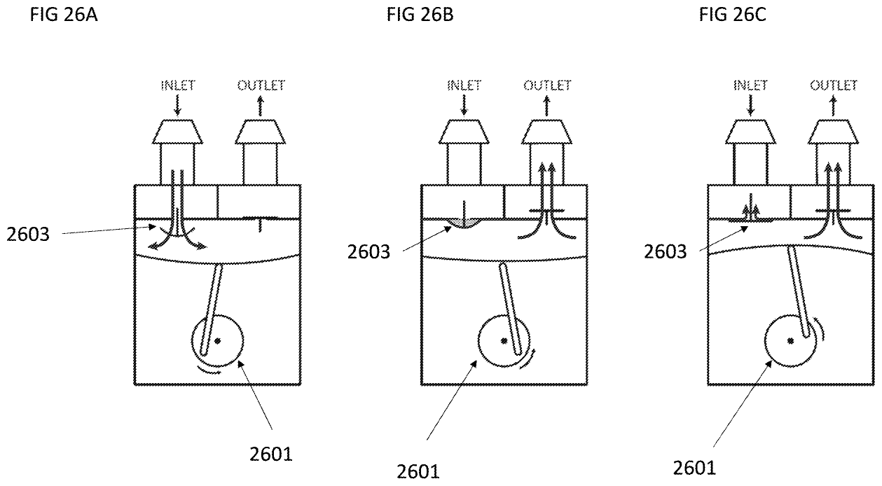

Referring now to FIGS. 26A, 26B, and 26C, another example embodiment of a vacuum motor device breast pump is shown. In this example, the vacuum motor device breast pump minimizes an amount of suction loss associated with production of the vibratory waveform. In other words, the vacuum motor device produces a vibratory waveform with a (slight) uptick, without sacrificing loss of a maximum achievable vacuum pressure.

In this embodiment, the example vacuum motor device breast pump includes a single vacuum motor 2601 to leverage the inherent step-wise waveform associated therewith. Determination of the ideal vibration frequency would allow for the selection of the appropriate size of the motor to achieve the desired rate of change in the vacuum.

To introduce the uptick that is synchronized to the upstroke of the vacuum motor 2601, a controlled-leak flap valve 2603 is positioned at an inlet side. One potential embodiment of this controlled-leak flap valve 2603 is a concave-flap configuration with a cavity. During the upstroke phase of the vacuum motor 26C, the air within the concave-flap cavity 2603 would be expelled back into the system resulting in a controlled amplitude of the uptick. Since the air in the cavity of flap valve 2603 comes from the inlet side rather than a leak to an outlet side, any loss in pressure associated with the vibratory effect caused by the expelled air is minimized.

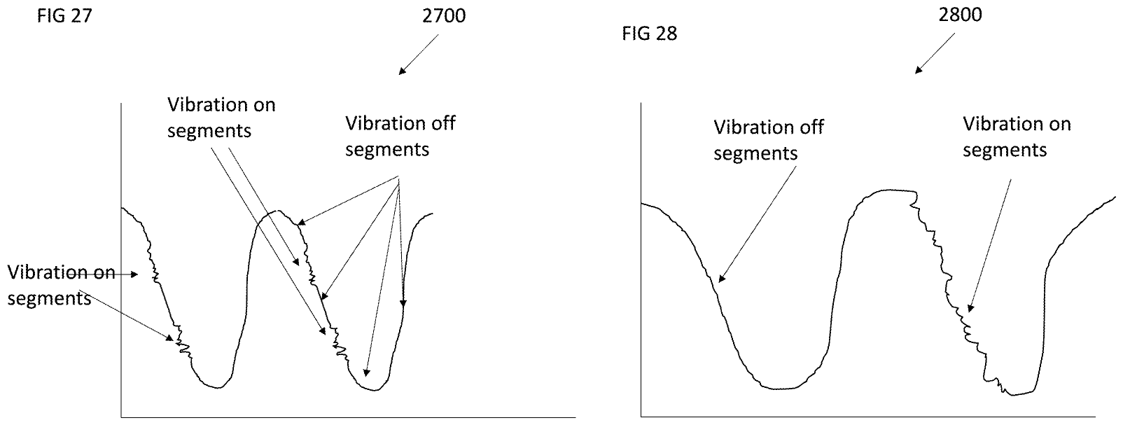

Referring now to FIGS. 27-29, the embodiments described herein can be configured to generate a variety of vibratory waveforms that can be optionally tuned by the user.

For example, FIG. 27 illustrates a vibratory waveform 2700 for a breast pump in which vibration is introduced periodically at two segment of each downslope and turned off on each subsequent upslope.

FIG. 28 illustrates an alternative vibratory waveform 2800 that includes vibration that is introduced at different periods, such as every other downslope.

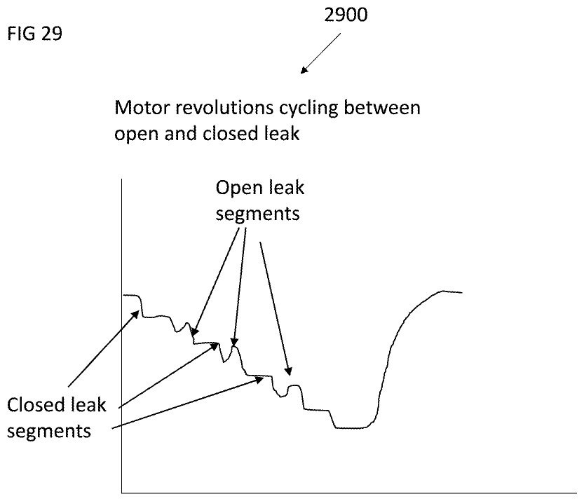

Finally, FIG. 29 illustrates another vibratory waveform 2900 that includes oscillating once per cycle. This could be accomplished by rotation that causes a valve to open or close on alternating revolutions of the motor.

Many other configurations for the vibratory waveforms can be accomplished according to the embodiments described herein.

In one aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction for extracting the milk; an electromechanical device configured to selectively allow air into the inlet portion or the outlet portion to create a vibratory waveform; and a controller programmed to receive input from the user to control the electromechanical device to thereby manipulate the vibratory waveform.

In another aspect, a vacuum motor device for facilitating milk extraction from a breast of a user includes: an inlet portion; an outlet portion coupled to the inlet portion; a first motor configured to cause air to flow into the inlet portion and out of the outlet portion during a breast pumping cycle to create suction for extracting the milk; a second motor configured to selectively allow air into the inlet portion or the outlet portion to create a vibratory waveform; and a controller programmed to receive input from the user to control the second motor to thereby manipulate the vibratory waveform.

In another aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction for extracting the milk; a first electromechanical device configured to selectively allow air into the inlet portion or the outlet portion to create a vibratory waveform; a second electromechanical device configured to selectively allow air into the inlet portion or the outlet portion to create a vibratory waveform; and a controller programmed to receive input from the user to control one or both of the first electromechanical device and the second electromechanical device to thereby manipulate the vibratory waveform.

In yet another aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction for extracting the milk; an electromechanical device configured to selectively block the suction to create a vibratory waveform; and a controller programmed to receive input from the user to control the electromechanical device to thereby manipulate the vibratory waveform.

In another aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction for extracting the milk; a first electromechanical device configured to selectively allow air into the inlet portion or the outlet portion; a second electromechanical device configured to selectively block the suction; and a controller programmed to receive input from the user to control one or both of the first electromechanical device and the second electromechanical device to thereby manipulate a vibratory waveform.

In another aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction in a suction tube for extracting the milk; a restricting device configured to selectively engage the suction tube to create a vibratory waveform; and a controller programmed to receive input from the user to control the restrictive device to thereby manipulate the vibratory waveform.

In yet another aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction for extracting the milk; an opening formed in the device to allow air into the inlet portion or the outlet portion to create a vibratory waveform; a fastener sired to engage the opening; and a controller programmed to receive input from the user to thread the fastener into or out of the opening to thereby manipulate the vibratory waveform.

In another aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction for extracting the milk; a bleeder valve configured to selectively allow air into the inlet portion or the outlet portion based upon a pressure exerted by the air on the bleeder valve to create a vibratory waveform; and a controller programmed to receive input from the user to control the bleeder valve to thereby manipulate the vibratory waveform.

In another aspect, a vacuum motor device for facilitating milk extraction from a breast of a user includes: an inlet portion; an outlet portion coupled to the inlet portion; a motor configured to cause air to flow into the inlet portion and out of the outlet portion during a breast pumping cycle to create suction for extracting the milk; a flap valve defining a cavity, the flap valve being configured to: open to allow air into the inlet portion or out of the outlet portion during a first portion of the breast pumping cycle; close to stop air from going into the inlet portion or out of the outlet portion during a second portion of the breast pumping cycle; and expel air from the cavity into the inlet portion or out of the outlet portion during a third portion of the breast pumping cycle.

Although this detailed description has set forth certain embodiments and examples, the present invention extends beyond the specifically disclosed embodiments to alternative embodiments and/or uses of the invention and modifications and equivalents thereof. Thus, it is intended that the scope of the present invention should not be limited by the particular disclosed embodiments described above.

We claim:

1. A vacuum motor device for facilitating milk extraction from a breast of a user, the device comprising:
an inlet portion;
an outlet portion coupled to the inlet portion;
a motor configured to cause air to flow into the inlet portion and out of the outlet portion during a breast pumping cycle to create suction for extracting the milk;
an electromechanical device configured to selectively allow air into the inlet portion or the outlet portion to create a vibratory waveform; and
a controller programmed to control the electromechanical device.

2. The vacuum motor device of claim 1, wherein the electromechanical device is a second motor configured to selectively allow air into the inlet portion or the outlet portion to create the vibratory waveform.

3. The vacuum motor device of claim 1, further comprising:
a second electromechanical device configured to selectively allow air into the inlet portion or the outlet portion to create the vibratory waveform;
wherein the controller is programmed to receive input from the user to control one or both of the electromechanical device and the second electromechanical device to thereby manipulate the vibratory waveform.

4. The vacuum motor device of claim 1, wherein a frequency of the vibratory waveform is between 2 Hz and 20 Hz.

5. The vacuum motor device of claim 1, further comprising an h-bridge to drive the motor to cyclically create a vacuum and release the vacuum, by alternating a polarity to the motor.

6. The vacuum motor device of claim 1, wherein the electromechanical device is a second motor configured to create the vibratory waveform by increasing and decreasing pressure.

7. The vacuum motor device of claim 1, wherein the electromechanical device includes a solenoid that is modulated to provide the vibratory waveform.

8. The vacuum motor device of claim 7, wherein the electromechanical device includes multiple solenoids configured to release a vacuum and provide the vibratory waveform.

9. The vacuum motor device of claim 7, wherein the solenoid is positioned in a normally-opened configuration or a normally-closed configuration.

10. The vacuum motor device of claim 1, further comprising a feedback control mechanism configured to tune the vibratory waveform.

11. A vacuum motor device for facilitating milk extraction from a breast of a user, the device comprising:
an inlet portion;
an outlet portion coupled to the inlet portion;
a motor configured to cause air to flow into the inlet portion and out of the outlet portion during a breast pumping cycle to create suction for extracting the milk;
an electromechanical device configured to selectively block the suction to create a vibratory waveform; and
a controller programmed to receive input from the user to control the electromechanical device to thereby manipulate the vibratory waveform.

12. The vacuum motor device of claim 11, further comprising:
a second electromechanical device configured to selectively block the suction;
wherein the controller is programmed to receive input from the user to control one or both of the electromechanical device and the second electromechanical device to thereby manipulate the vibratory waveform.

13. The vacuum motor device of claim 11, further comprising:
a suction tube for extracting the milk; and
a restricting device configured to selectively engage the suction tube to create the vibratory waveform.

14. The vacuum motor device of claim 13, wherein the controller is programmed to receive input from the user to control the restricting device to thereby manipulate the vibratory waveform.

15. The vacuum motor device of claim 11, further comprising:
an opening formed in the device to allow air into the inlet portion or the outlet portion to create the vibratory waveform; and
a fastener sized to engage the opening.

16. The vacuum motor device of claim 15, wherein the controller is programmed to receive input from the user to thread the fastener into or out of the opening to thereby manipulate the vibratory waveform.

17. The vacuum motor device of claim 11, further comprising a bleeder valve configured to selectively allow air into the inlet portion or the outlet portion based upon a pressure exerted by the air on the bleeder valve to create the vibratory waveform.

18. The vacuum motor device of claim 17, wherein the controller is programmed to receive input from the user to control the bleeder valve to thereby manipulate the vibratory waveform.

19. A vacuum motor device for facilitating milk extraction from a breast of a user, the device comprising:
an inlet portion;
an outlet portion coupled to the inlet portion;
a motor configured to cause air to flow into the inlet portion and out of the outlet portion during a breast pumping cycle to create suction for extracting the milk;
a flap valve defining a cavity, the flap valve being configured to:
open to allow air into the inlet portion or out of the outlet portion during a first portion of the breast pumping cycle;
close to stop air from going into the inlet portion or out of the outlet portion during a second portion of the breast pumping cycle; and
expel air from the cavity into the inlet portion or out of the outlet portion during a third portion of the breast pumping cycle.
